(19) United States
(12) Patent Application Publication
(10) Pub. No.: US 2026/0146974 A1
(43) Pub. Date: May 28, 2026

(54) Acoustic Windows with Limited Acoustic Attenuation for Ultrasound Probes

(71) Applicant: BFLY Operations, Inc, Burlington MA, MA (US)

(72) Inventors: Simon Esteve, Guilford, CT (US); Jaime Scott Zahorian, Guilford CT, CT (US); Nikhil Apte, San Jose, CA (US); Farah Qureshi, Sunnyvale, CA (US); Sarp Satir, Branford, CT (US)

(21) Appl. No.: 18/864,272

(22) PCT Filed: May 9, 2023
(86) PCT No.: PCT/US2023/021508

§ 371 Date: Nov. 8, 2024

Related U.S. Application Data
(60) Provisional application No. 63/339,835, filed on May 9, 2022.

(51) Int. Cl. G01N 29/28; G01N 29/24

(52) U.S. Cl. CPC G01N 29/28; G01N 29/2406; G01N 29/2437; G01N 2291/02466; G01N 2291/02483

(57) ABSTRACT

An ultrasound probe includes an ultrasound transducer stack comprising one or more ultrasound transducers emitting an acoustic signal, and an acoustic window passing the acoustic signal. The acoustic window is composed of a butadiene-based compound. In one or more embodiments, the acoustic window is made of butadiene rubber. Butadiene rubber has acoustic characteristics that may be suitable for acoustic windows, as subsequently discussed. Specifically, the use of butadiene rubber may result in desirable acoustic attenuation, acoustic velocity and acoustic impedance.

### CROSS-REFERENCE TO RELATED APPLICATIONS

This application claims the benefit of priority under 35 U.S.C. § 119(e) to U.S. Provisional Patent Application Ser. No. 63/339,835, filed on May 9, 2022, which is hereby incorporated by reference herein in its entirety.

### BACKGROUND

An ultrasound probe may include multiple ultrasound transducers arranged in a transducer array that emits ultrasound signals. The ultrasound signals may be reflected by body tissue thereby resulting in an echo. The ultrasound transducers may receive the echo as a received ultrasound signal, and the received ultrasound signal may be processed to generate an ultrasound image or sonogram.

Certain acoustic characteristics of the acoustic window may be necessary or desirable in order to achieve satisfactory performance of the ultrasound probe. For example, a certain focusing characteristic may necessitate a certain ness of the acoustic window; the acoustic attenuation may be required not exceed a certain value; the impedance may be required to closely match the impedance of tissue; etc.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to an ultrasound probe, comprising: an ultrasound transducer stack comprising one or more ultrasound transducers emitting an acoustic signal; and an acoustic window passing the acoustic signal, wherein the acoustic window is composed of a butadiene-based compound.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows an ultrasound imaging scenario in accordance with one or more embodiments.

FIG. 2 schematically shows a cross-sectional view of an ultrasound probe in accordance with one or more embodiments.

FIGS. 3A, 3B, 3C, and 3D show different views of elements of an ultrasound probe in accordance with one or more embodiments.

FIG. 4 shows an acoustic window in accordance with one or more embodiments.

FIG. 5 schematically shows an implementation example of an ultrasound system integrated on a chip, in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure relate to acoustic windows with limited acoustic attenuation and ultrasound probes equipped with such acoustic windows.

An ultrasound transducer array may be equipped with an acoustic window. The acoustic window may couple the acoustic signal to and from the ultrasound transducers.

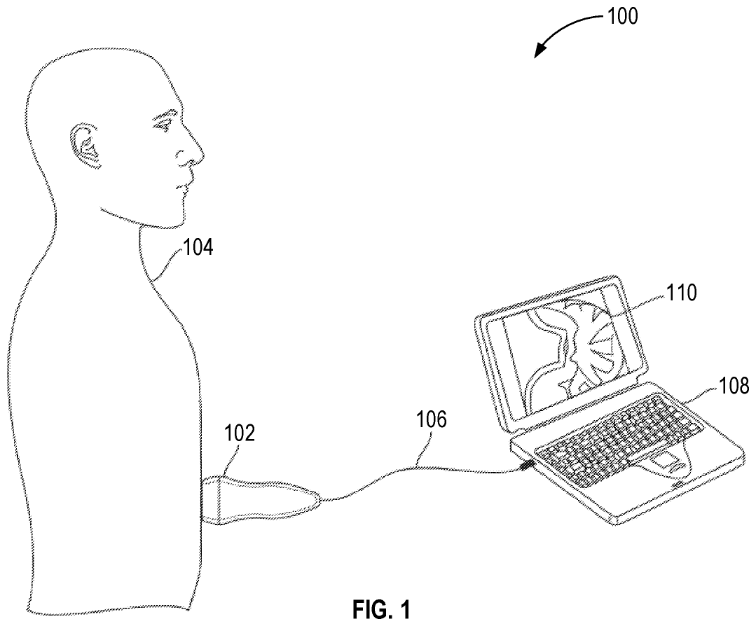

FIG. 1 shows an example of an ultrasound imaging scenario in accordance with one or more embodiments. The ultrasound imaging scenario (100) illustrates the use of an ultrasound probe (102) to obtain ultrasound images (sonograms) from an imaging subject (104). Data collected by the ultrasound probe (102) may be transmitted to one or more external computer devices (108) for further processing. For example, ultrasound probe (102) may transmit the data via a wired or wireless connection (106) to a computer device (108) (a laptop in this non-limiting example), which may process the data to generate and display an image (110) of the imaging subject (104) on a display.

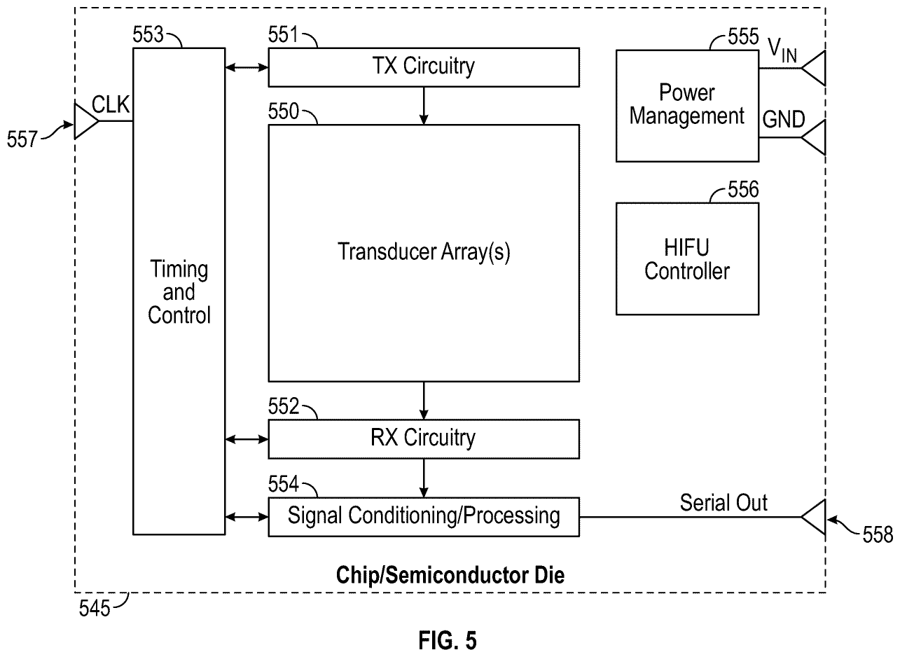

The ultrasound probe (102) may include various components that enable the transmission and/or reception of acoustic waves, as subsequently discussed. The components may be arranged in different manners, without departing from the disclosure. For example, various components of the ultrasound probe (102) may be integrated on-chip. Alternatively, discrete components of partially integrated components may be used. An example of a configuration that includes ultrasound transducers as well as ultrasound circuitry integrated on a chip is described below in reference to FIG. 5.

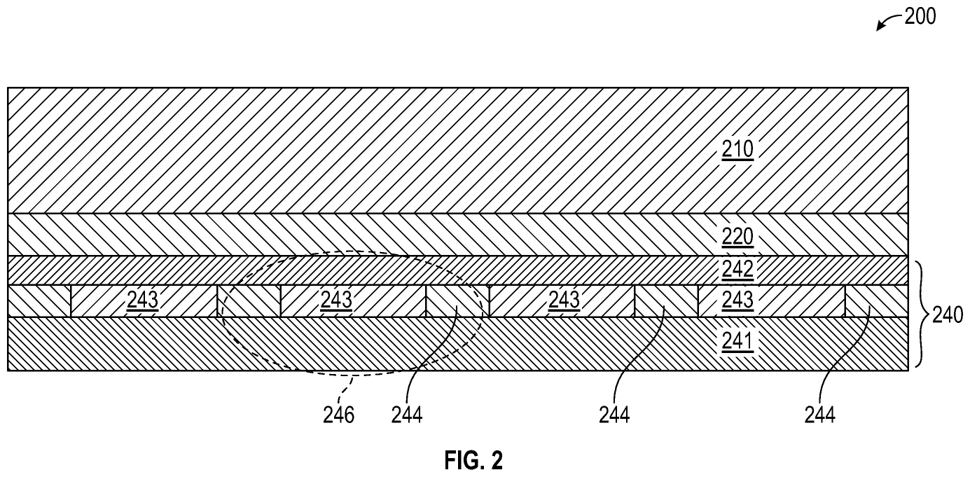

FIG. 2 shows a simplified cross-sectional view of an ultrasound probe (200) in accordance with one or more embodiments. The ultrasound probe (200) may include an acoustic window (210) and an ultrasound transducer stack (240). The ultrasound probe (200) may further include a coupling layer (220) (e.g., a boundary layer) disposed between the ultrasound transducer stack (240) and the acoustic window (210). While FIG. 2 shows certain elements, the ultrasound probe may include additional elements, without departing from the disclosure. In one or more embodiments, ultrasound transducers (246) are formed by elements arranged in the ultrasound transducer stack (240). The ultrasound transducers (246) may be arranged in a transducer array which may be integrated on a single semiconductor die. In the example shown in FIG. 2, the transducer stack (240) includes a substrate (241), a membrane (242), and cavity sidewalls (244) which enclose cavities (243). In the area of each of the cavities (243), the membrane (242) may vibrate, thus forming an ultrasound transducer (246). The ultrasound transducers (246) may be used to transduce an acoustic signal into an electric signal, or vice versa. Silicon materials may be used for the substrate (241), the membrane (242), and/or the cavity sidewalls (244), and the ultrasound transducers (246) may be on a chip.

In one or more embodiments, the ultrasound transducers (246) formed in the ultrasound transducer stack (240) are Capacitive Micromachined Ultrasonic Transducers (CMUTs) in which the cavities (243) are micromachined. A more detailed description may be found in, for example, U.S. Pat. No. 9,067,779, and U.S. patent application Ser. No. 16/296,476 which are hereby incorporated by reference in their entirety, While not shown, the substrate (241) may also accommodate integrated circuity used for driving and/or interrogating the ultrasound transducers (246).

Also, the transducer stack (240) may include other components, e.g., a heat spreader for cooling the chip with the transducers, a printed circuit board that accommodates the chip with the transducers, etc.

In one or more embodiments, the acoustic window (210) is made of butadiene rubber. Butadiene rubber has acoustic characteristics that may be suitable for acoustic windows, as subsequently discussed. Specifically, the use of butadiene rubber may result in desirable acoustic attenuation, acoustic velocity and acoustic impedance.

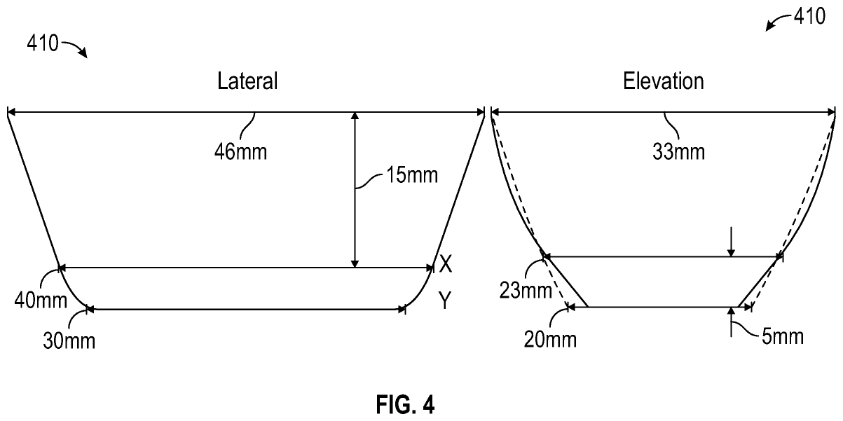

In one or more embodiments, an acoustic window has a limited acoustic attenuation. For example, an unfilled butadiene rubber with a high-cis microstructure may have an acoustic attenuation of ˜5 dB/cm at 5 MHz. The limited attenuation may enable the use of thicker windows. The use and application of thicker windows is discussed below in reference to FIG. 4.

In one or more embodiments, an acoustic window is non-defocusing. More specifically, an acoustic window in accordance with embodiments of the disclosure passes acoustic signals (neither focusing nor defocusing acoustic signals). Focusing behavior of an acoustic window is governed by the speed of sound in the acoustic window vs the speed of sound in tissue. In particular, in one or more embodiments, an acoustic window (220) has a speed of sound c₁ approximately equal to the speed of sound c₂ in tissue (c₁=c₂). A typical speed of sound in tissue is ˜1600 m/s. An unfilled butadiene rubber with a high-cis microstructure may have a speed of sound of ˜1560 m/s, thus being approximately the speed of sound in tissue. As discussed below in reference to FIG. 4, an acoustic window with a limited-size footprint (e.g., small enough to perform cardiac imaging between the ribs) may be designed when the window has at least a certain thickness.

In one or more embodiments, an acoustic window has an acoustic impedance that limits reflections at the window-tissue interface. Reflections at the window-tissue interface may be reduced by matching the impedance of the acoustic window with the impedance of the tissue. The acoustic impedance of a material is the product of the speed of sound in a material and the density of the material. The impedance of tissue may be ˜1.5-1.6 Mrayl. An unfilled butadiene rubber with a high-cis microstructure may have a density of 0.95 g/cm³, thereby resulting in an impedance close to the impedance of tissue.

Butadiene rubber, in an uncured state, may not be very stable and may undergo changes over time, such as hardening when exposed to heat, UV or oxygen (ozone). To become stable the butadiene needs to be compounded with other ingredients such as antioxidants, vulcanization and crosslinking agents, oil additive, catalyzer and fillers which impact its elastomeric properties.

A butadiene-based compound that may be used as an acoustic window may need to meet some criteria, such as wear resistance, chemical resistance for cleaning and disinfection, and biocompatibility for skin contact and cosmetic aspects, while maintaining the acoustic characteristics (acoustic attenuation, acoustic velocity and acoustic impedance).

In one or more embodiments, the butadiene-based compound used for the acoustic window incorporates carbon black filler to improve the wear resistance, increase the hardness and, as an additional benefit, to give a uniform black color to the window surface.

The addition of carbon black fillers may impact the acoustic attenuation by diffraction of the ultrasound wave depending on the size of the filler with respect to the acoustic wavelength. Depending on the application, a minimum amount of acoustic attenuation can also be beneficial to avoid unwanted reflection from the window-tissue boundary. The amount and size of filler allows for the ability to tune the acoustic attenuation of the acoustic window. As an example, the incorporation of ˜10% of Carbon Black of an average size of 45 μm which is <to ⅓ of the wavelength at 10 MHz yields an attenuation of ˜5 dB/cm at 5 MHz and ˜15 dB/cm at 10 MHz. If a lower attenuation is required or desired at higher frequency, finer filler may be used. To facilitate the integration of the carbon black filler into the butadiene during the milling process, some ˜5% oil is added to the developed compound.

In one or more embodiments, the density and speed of sound of the butadiene-based compound does not differ significantly from the unfilled butadiene rubber which maintains a good impedance match with tissue.

An alternative to the butadiene rubber material, PEBAX 2533 SA 01 MED, which may have similar characteristics, may be used.

The acoustic window may be fabricated by direct molding or transfer molding processes. The ultrasound transducer array may then be coupled to the acoustic window through various methods, e.g., gluing with a soft epoxy layer. In some applications the acoustic window may be directly overmolded onto the probe housing to simplify further the manufacturing integration.

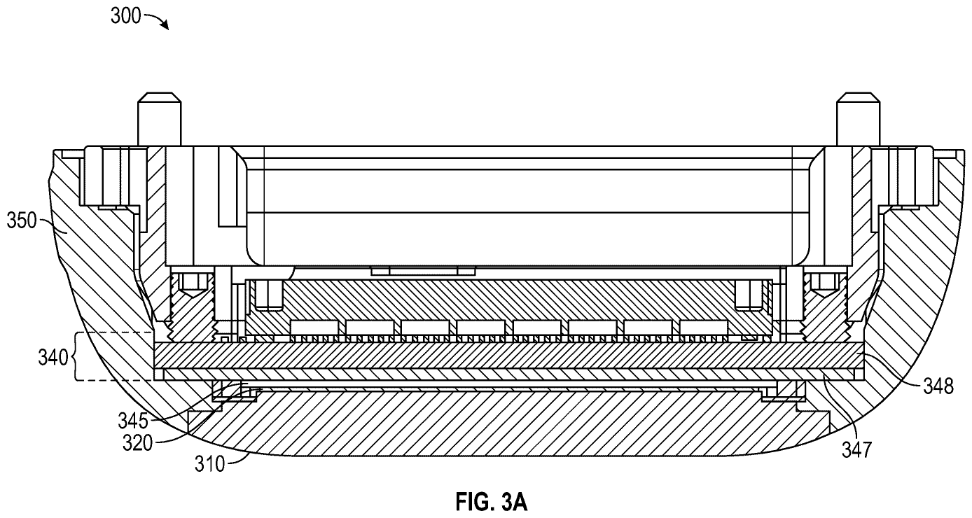

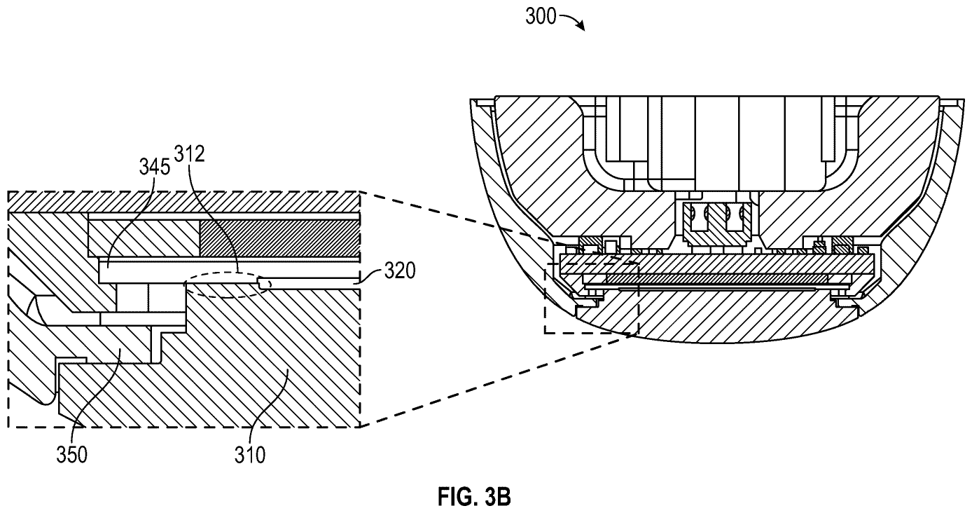

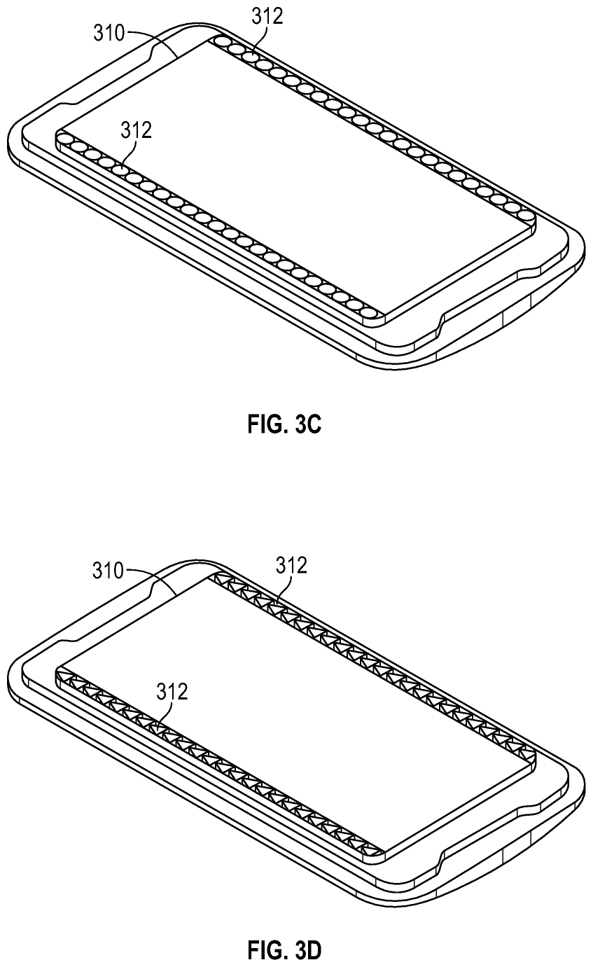

Turning to FIGS. 3A-3D, FIG. 3A shows elements of an ultrasound probe in accordance with one or more embodiments. The ultrasound probe (300) includes a shroud (350), an ultrasound transducer stack (340), an acoustic coupling layer (320), and an acoustic window (310). In the example of FIG. 3A, the ultrasound transducer stack (340) includes various elements such as the chip (345) with the ultrasound transducers, the heat spreader (347) and the printed circuit board (348), as previously described. While FIGS. 3A-3D show certain elements, the ultrasound probe may include additional elements, without departing from the disclosure. The shroud (350) houses the elements of the ultrasound probe (300) and may acoustically, thermally (e.g., acting as a heat sink), and/or mechanically (e.g., providing structural rigidity) protect the ultrasound transducer stack (340). The shroud (350) may be formed from the same material as the body of the ultrasound probe (300), e.g., aluminum, plastic, a composite material, etc.

FIGS. 3B-3D provide additional views of elements of an ultrasound probe in accordance with one or more embodiments. Standoffs (312) are added on the backside (facing the chip (345)) of the acoustic window (310). Each standoff (312) may be a raised portion or protrusion of the acoustic window (310). As shown in FIG. 3B, the standoffs (312) may be in mechanical contact with an inactive area of the chip (345) (i.e., an area not involved in the emission/reception of acoustic waves). The standoffs (312) may ensure that there is an acoustic coupling layer or glue layer (320) of a specified thickness between the acoustic window (310) and the chip (345), whereas the mechanical flexibility of the acoustic window (310) ensures that the transducer stack (340) including the chip (345) is in a defined mechanical position relative to the shroud (350). In other words, during mechanical assembly of the ultrasound probe (300), the acoustic window (310) may deform until the transducer stack (340) hard-stops on the shroud (350). The acoustic coupling layer (320) may have damping characteristics to reduce surface waves. The acoustic coupling layer (320) may be a continuous surface, or a complex, patterned surface as defined by the mold tooling used for manufacturing. FIG. 3C shows an acoustic window (310) with circular standoffs (312), whereas FIG. 3D shows an acoustic window (310) with triangular standoffs (312). The standoffs, in the examples, are arranged along each of two edges of the acoustic window. The standoffs (312) may be complex shapes (e.g., polygons of any type) to help break up waves that travel along the surface and are reflected on the edges of the transducer array.

FIG. 4 shows a lateral view and an elevation view of an acoustic window (410), in accordance with embodiments of the disclosure. Ultrasound transducers have an active area and an associated maximum size in the X-Y dimensions based on the level of integration and packaging. In the case of a CMUT transducer array, integrated with CMOS electronics, it is possible that the ASIC and not the active acoustic area dictates the overall dimensions. To reduce the footprint of the tissue-facing front of the probe, it may be necessary to use a thicker window. FIG. 4 illustrates an example of how increasing the thickness of the window (e.g., by 5 mm) can result in reduction of the footprint (e.g., from 40×23 mm to 30×20 mm) both along the lateral and elevational dimensions. The reduced footprint may be helpful, for example, to fit between the ribs for improved cardiac imaging modalities while still being useful for whole body scanning.

An acoustic window made of a butadiene-based compound may provide enough thickness for the beamforming-based focusing through the acoustic window to achieve the desired reduction of the footprint. Despite the thickness of the acoustic window, attenuation does not critically affect performance because the butadiene-based compound has a relatively low attenuation. Further, the butadiene-based compound may help avoid unwanted reflection at the window-tissue interface, because the acoustic impedance of the window approximately matches the acoustic impedance of the tissue.

More generally, acoustic windows in accordance with embodiments of the disclosure allow for optimization of the transducer-tissue contact. With the speed of sound the same as the human body, complex shapes that do not distort the electronically enabled acoustic focusing (beamforming) of the transducer array. Further, with a significantly lower attenuation than traditional lens materials, it is possible to increase the thickness of the acoustic window as needed to achieve the desired shape without introducing excessive acoustic attenuation.

FIG. 5 schematically shows an implementation example of an ultrasound system integrated on a chip (545), in accordance with one or more embodiments. The example is provided for illustrative purposes only and is not intended to limit the scope of the disclosure. The chip (545) may include one or more transducer arrangements (e.g., transducer array (550)), transmit (TX) circuitry (551), receive (RX) circuitry (552), a timing and control circuit (553), a signal conditioning/processing circuit (554), a power management circuit (555), and/or a high-intensity focused ultrasound (HIFU) controller (556). In the embodiment as shown, all of the illustrated elements are formed on a single semiconductor die. In other embodiments, one of more of the elements may be discrete components. In addition, although the illustrated example shows both TX circuitry (551) and RX circuitry (552), in alternative embodiments only TX circuitry (551) or only RX circuitry (552) may be employed. For example, such embodiments may be employed in transmission-only ultrasound probes or reception-only ultrasound probes. The TX circuitry (551) may generate pulses to energize the individual elements of the transducer array (550) so as to emit an ultrasound pulse for imaging. Likewise, the RX circuitry (552) may receive and process electronic signals generated by the individual elements of the transducer arrays (550). In one embodiment, the chip (545) accommodates the transducer array (550) on a plain substrate, whereas the other components shown in FIG. 5 are located elsewhere.

The ultrasound transducers in the transducer array (550) may be arranged in various manners. In some embodiments, the transducer array (550) may include capacitive micromachined ultrasonic transducers (CMUTs), CMOS ultrasonic transducers (CUTS), piezoelectric micromachined ultrasonic transducers (PMUTs), and/or other suitable ultrasonic transducer cells. The timing and control circuit (553) may generate various timing and control signals that may be used to synchronize and coordinate the operation of the components on the chip (545). An input port (557) may provide a clock signal CLK to supply the timing to the control circuit (553). The signal conditioning/processing circuit (554) may generate a high-speed serial data stream which is outputted by one or more output ports (558). The high-speed serial data stream may include the data (e.g., received acoustic signals) obtained from the transducer array (550) via the RX circuitry (552). The power management circuit (555) may convert one or more input voltages VIN from an off-chip source into voltages needed to carry out operation of the chip. Likewise, the power management circuit (555) may manage power consumption of the components on the chip (545).

The HIFU controller (556) may generate one or more HIFU signals via one or more elements of the transducer arrays (550) to provide HIFU functionality to provide the transducer arrays (550) a power level appropriate for imaging applications.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

1. An ultrasound probe, comprising:
an ultrasound transducer stack comprising one or more ultrasound transducers that emit an acoustic signal; and
an acoustic window that passes the acoustic signal, wherein the acoustic window is composed of a butadiene-based compound; and
the acoustic window is secured to the ultrasound probe by an acoustic coupling layer.

2. The ultrasound probe of claim 1, wherein the butadiene-based compound comprises:
an unfilled butadiene rubber with a high-cis microstructure.

3. The ultrasound probe of claim 1, wherein the butadiene-based compound has an acoustic velocity that substantially matches the acoustic velocity of tissue.

4. The ultrasound probe of claim 1, wherein the butadiene-based compound has a density of approximately 0.95 g/cm³.

5. The ultrasound probe of claim 1, wherein the butadiene-based compound comprises a stabilizing agent including at least one of:
an antioxidant,
a vulcanization agent,
a crosslinking agent,
an oil additive,
a catalyzer.

6. The ultrasound probe of claim 1, wherein the butadiene-based compound comprises a carbon black filler.

7. The ultrasound probe of claim 1, wherein the acoustic window has an acoustic impedance that substantially matches an acoustic impedance of tissue.

8. The ultrasound probe of claim 1, wherein beamforming-based focusing characteristics are obtained by the acoustic window having a specified thickness where the specified thickness is enabled by attenuation characteristics of the acoustic window.

9. The ultrasound probe of claim 1, wherein a tissue-facing footprint based on the focusing characteristics of the acoustic window is no more than 30×20 mm.

10. The ultrasound probe of claim 89, wherein the specified thickness is 20 mm.

11. The ultrasound probe of claim 1, wherein the acoustic window comprises a plurality of standoffs arranged along each of at least two edges of the acoustic window.

12. The ultrasound probe of claim 11, wherein each of the standoffs is one of triangular, circular, or polygon-shaped.

13. (canceled)

14. The ultrasound probe of claim 1, wherein the
acoustic coupling layer is disposed between the acoustic window and the ultrasound transducer stack.

15. The ultrasound probe of claim 1,
wherein the one or more ultrasound transducers are at least one selected from a group consisting of a capacitive micromachined ultrasonic transducer (CMUT) and a piezoelectric micromachined ultrasonic transducer (PMUT).

16. The ultrasound probe of claim 1,
wherein the acoustic window neither substantially focuses nor substantially defocuses the acoustic signals.

17. The ultrasound probe of claim 1,
wherein a footprint of a portion of the acoustic window which is in contact with tissue is sufficiently small as to fit between the ribs of a human.

18. The ultrasound probe of claim 1,
wherein the coupling layer has a speed of sound which is less than 50% of the acoustic window.

19. A method of manufacturing an acoustic window comprising;
generating a butadiene-based compound by,
providing an unfilled butadiene rubber and adding at least one stabilizing agent and a carbon black filler, wherein the carbon black filler is approximately 10% of the butadiene-based compound, and
molding the butadiene-based compound into a rectangular acoustic window wherein,
standoffs are disposed on at least one edge of a backside of the acoustic window,
a tissue-facing footprint of the acoustic window is no more than 30×20 mm, and
an array-facing footprint of the acoustic window is approximately 40×23 mm.

20. The method of claim 19 where the acoustic window is fabricated by direct molding or transfer molding.

21. The method of claim 19 where the acoustic window is fabricated by directly overmolding the window onto the probe housing.

22. The method of claim 19 wherein the standoffs define the thickness of an acoustic coupling layer applied to the backside of the acoustic window.
